(19) United States
(12) Patent Application Publication
(10) Pub. No.: US 2011/0268776 A1
(43) Pub. Date: Nov. 3, 2011

(54) PROGRAMMED-RELEASE, NANOSTRUCTURED BIOLOGICAL CONSTRUCT FOR STIMULATING CELLULAR ENGRAFTMENT FOR TISSUE REGENERATION

(76) Inventors: Jay N. Schapira, Beverly Hills, CA (US); Raj Makkar, Los Angeles, CA (US)

(21) Appl. No.: 13/161,459

(22) Filed: Jun. 15, 2011

Related U.S. Application Data
(63) Continuation-in-part of application No. 12/150,329, filed on Apr. 25, 2008.
Application No. 12/150,329 is a continuation of application No. 12/221,139, filed on Jul. 31, 2008, now abandoned.
(60) Provisional application No. 60/926,306, filed on Apr. 25, 2007.
(60) Provisional application No. 60/931,749, filed on May 25, 2007.
(60) Provisional application No. 60/935,021, filed on Jul. 20, 2007.
(60) Provisional application No. 60/963,290, filed on Aug. 3, 2007.
(60) Provisional application No. 60/963,290, filed on Aug. 3, 2007.

(51) Int. Cl. A61K 9/00; B82Y 5/00

(52) U.S. Cl. 424/400; 977/906

(57) ABSTRACT

A biologically engineered construct comprising of a polymeric biomatrix, designed with a nanophase texture, and a therapeutic agent for the purpose of tissue regeneration and/or controlled delivery of regenerative factors and therapeutic substances after it is implanted into tissues, vessels, or luminal structures within the body. The therapeutic agent may be a therapeutic substance or a biological agent, such as antibodies, ligands, or living cells. The nanophase construct is designed to maximize lumen size, promote tissue remodeling, and ultimately make the implant more biologically compatible. The nano-textured polymeric biomatrix may comprise one or more layers containing therapeutic substances and/or beneficial biological agents for the purpose of controlled, physiological, differential substance/drug delivery into the luminal and abluminal surfaces of the vessel or lumen, and the attraction of target molecules/cells that will regenerate functional tissue. The topographic and biocompatible features of this layered biological construct provides an optimal environment for tissue regeneration along with a programmed-release, drug delivery system to improve physiological tolerance of the implant, and to maximize the cellular survival, migration, and integration within the implanted tissues.

### CROSS-REFERENCES TO RELATED APPLICATIONS

This application is a continuation-in-part of U.S. application Ser. No. 12/150,329, filed Apr. 25, 2008, which claims the benefit of U.S. Provisional Application Nos. 60/926,306, filed Apr. 25, 2007; 60/931,749, filed May 25, 2007; 60/935,021, filed Jul. 20, 2007; and 60/963,290, filed Aug. 3, 2007. This application is also a continuation of U.S. application Ser. No. 12/221,139, filed Jul. 31, 2008, which claims the benefit of U.S. Provisional Application Nos. 60/935,021, filed Jul.20, 2007; and 60/963,290, filed Aug. 3, 2007. Each of the foregoing applications is incorporated herein by this reference.

### FIELD OF THE INVENTION

The present invention relates to the use of a biologically engineered construct that will be used for tissue regeneration and controlled drug delivery after it is implanted into tissues, vessels, or luminal structures within the body.

### BACKGROUND OF THE INVENTION

Each year, millions of patients undergo the implantation of a medical device or medication delivery system into the eye, vessels, organs, bone, cartilage, flesh, ducts and/or luminal structures within the body for the treatment of various diseases and the complications associated with these diseases. The cyto-compatibility of these implants is still imperfect, however. Implantation is often accompanied by a risk of biological rejection, cellular migration, impaired, undesirable, and excessive tissue healing, clot development on the device surface, or infection. This problem has limited the application of the currently available implantable biomaterials, drug-delivery technology, and cell therapy strategies.

Implantation upsets the organic systems physiology of the host tissue. Following device placement, the tissue becomes a hostile environment for cellular function and subsequent tissue regeneration. Regardless of the organ or tissue type, these injuries inevitably disrupt the fine balance of cellular signaling, differentiation, proliferation, and death. The majority of tissues are heterogeneous, that is, they are comprised of several different cell types that thrive based on cell-to-cell communication. These chemical signals are crucial for cellular survival, and are greatly disrupted by the introduction of a therapeutic device. The natural healing process is impeded and can often be further complicated by age and disease state. The current invention provides a method of controlled substance delivery following device placement that will mimic the physiological healing process thereby making implants more biocompatible, and improving overall healing.

In the field of tissue engineering, physicians and scientists have encountered numerous problems with poor osteoblast adhesion and osteointegration following bone implant surgeries. Similarly, bladder and tissue implants have been problematic, as the un-seeded, or bare polymeric scaffolds used to regenerate “new” tissue, while promising, have demonstrated issues with cyto-compatibility, toxicity, and infection following placement. This is true of skin and wound-healing implements as well. In vascular applications, neo-intimal proliferation is a normal response following device implantation. It is comprised of smooth muscle cell proliferation and re-endothelialization of the implant. This response essentially “indigenizes” the device, but, in 25-30% of situations, smooth muscle cell proliferation becomes excessive, and results in re-stenosis of the vascular device. These complications invariably extend to any organ system following device implantation, as they are perceived as foreign bodies by the human immune system.

Many implantable devices have attempted to mitigate bio-rejection by utilizing polymers as drug carriers or biofilms, such as poly(l-lactic acid) (“PLA”), poly(glycolic acid) (“PGA”), poly (lactic-co-glycolic acid) (“PLGA”), polycaprolactone, poly(ether urethane), Dacron, polytetrafluorurethane, and polyurethane (“PU”). These polymers have shown some success in large arteries, bone, and dental applications, but their surface features are not optimal and, as they degrade, they are known to be thrombogenic in applications such as small diameter vessel grafts.

Considering this, the existing implants designed to improve both biocompatibility and healing demonstrate promise, but they fail to address the critical design issues of the device: 1) the need for surface topography that mimics the native biological environment of the tissue and 2) an implant that is able to recreate the spatial and temporal aspects of the physiological healing process of specific tissues.

The implantation of any therapeutic medical device immediately changes the specific tissue surface topography from nano-scale to micro-scale. Surface features on existing implantable medical devices having micro-scale resolution, and not nano-scale resolution, have proven to be inadequate, and those applications that have attempted nano-topography are generally directed at texturing the non-polymeric portion of the construct, which in many cases, is not exposed. As a result, the surface topography of the currently available implantable medical devices and/or polymers does not mimic a natural environment, limits organic bio-interaction, and does not create a suitable cellular environment for tissue regeneration. Because the natural surface texture of most tissues (eye, bone, neural, bladder, organ, and intimal vascular tissue) is nanoscale (up to 100 nm) in size, recent efforts have been dedicated to improving tissue regeneration by designing biocompatible devices with nano-scale surface features.

Successful implantation depends on careful replication of the cells' natural physiological and topographical environment. This includes mimicry of the composition, architecture, and surface texture of the construct. Surface chemistry (such as charge, hydrophilicity, hydrophobicity, protein adsorption) and topography (such as surface area and nano-phase surface) significantly effect how and where cells attach to biomaterials. A number of studies have demonstrated that the nanotopographic cues of biomaterials can significantly improve cellular responses and healing both directly and indirectly. This is believed to be partially due to the fact that nano-surfaces have perhaps 40% more surface area in the Z plane and are more hydrophilic in nature. The increase in surface area in a third dimension increases device-tissue adhesion. Nanophase surface properties favor protein adsorption and interaction. Proteins contained in extracellular matrices (fibronectin, laminin, vitronectin) are nano-structured (2-70 nm) and are accustomed to interacting with nanophase surfaces, thus the adsorption of these proteins will subsequently attract endothelial progenitor cells and other reconstructive factors, stimulate healing, and can better reconstitute the injured tissue.

The latest advances in the construction of biomaterials and novel classes of biodegradeable and non-biodegradable polymers have demonstrated that materials with nanoscale surface features can better support cellular responses in vascular, bone, neural, and bladder tissue applications. Novel nanophase polymers are both compliant and cyto-compatible, as they possess the key design parameter for biocompatibility; specifically, optimal topography. More specifically, results from these studies have provided the first evidence that the surface properties of nanotextured materials and polymers preferentially enhance the competitive adhesion of endothelial cells versus vascular smooth muscle cells when compared to conventional materials. Furthermore, stem cells, when combined with nanofibers placed in the rat brain, have been shown to reverse stroke-induced neural tissue damage. There also appears to be decreased macrophage, fibroblast, B-cell, and T-cell growth on nano-surfaces, making them inherently anti-inflammatory. While much of this information is based on results from in-vitro experiments and animal studies, there is great potential to extend the existing technology to implantable medical devices for permanent or semi-permanent use in human physiological systems.

In addition to the favorable surface properties provided by nano-textured materials, the biocompatibility of implanted devices can be amplified by the addition of biologically engineered “cell sheets.” The goal of engineering cell sheets is to create a functional, differentiated tissue ex-vivo that can later be transplanted into tissues and structures within the body. By seeding cells into a biodegradable scaffold, intact cell sheets, along with their deposited extra-cellular matrices can be can be harvested and transplanted into host tissues to promote regeneration (the scaffold can also be eliminated by layering the cell sheets, creating a three-dimensional, nano-textured tissue construct).

Another distinct advantage of the current invention is that it can be programmed to mimic the cellular events that take place in the physiological healing process. The thickness, composition (substance density) and degradation of the nano-textured polymeric material can be carefully controlled to expose functional portions of the polymer (and therapeutic agents seeded within), allowing for controlled substance delivery. Thus, the “programmable” nature of the device can be used for temporal, qualitative, and quantitative release of therapeutic agents in a manner that recapitulates the organic phases of the healing process of specific tissues tissue.

Previous inventors have proposed therapeutic, substance-filled, biocompatible polymers as well as the addition of nano-structures directly to surgical tools and implantable medical devices. It is not believed, however, that any prior art form has focused on combining these two ideas with the goal of improving the healing process with a controlled substance release system that is not only well tolerated and integrated by the tissues, but can also be designed to carefully re-create the physiological processes that occur during natural tissue regeneration. Neither nano-textured devices, nor seeded theurapeutic polymers can accomplish this alone, therefore there is still a need for implantable medical devices designed with optimal (nanophase) surface features that are well tolerated by the body, beneficicial for the tissues, and capable of re-creating the physiological processes and cellular cues observed in-vivo.

### SUMMARY OF THE INVENTION

The goal of this novel, “programmable” invention is to provide a method and a biological construct for addressing the problem of poor biological and physiological tolerance following medical device placement by adding a nanophase surface texture to an implantable device that is capable of temporal, qualitative, and quantitative elution of therapeutic agents in a manner that mimics the natural healing process of specific tissues.

The unique biological construct for improved, timed-release drug delivery and tissue remodeling following implantation, comprises a layered polymeric biomatrix, either with or without a polymeric bioscaffold having a nanophase surface texture designed to mimic the specific extracellular matrix of a tissue into which the polymer is implanted to improve the biocompatibility of the biological construct; and various therapeutic agents seeded within the polymeric biomatrix to promote positive tissue remodeling and organ function through controlled drug delivery, optimized cyto-compatible surface characteristics, favorable protein adsorption, and improved cellular interaction. The therapeutic agent may be a therapeutic substance such as a drug, chemical compound, biological compound, or a living cell.

### BRIEF DESCRIPTION OF THE DRAWINGS

FIG. 1 illustrates the formation of the biological construct of the present invention;

FIG. 2 shows a cross-section of an embodiment of the biological construct of the present invention;

FIG. 3 illustrates another embodiment of the formation of the biological construct of the present invention;

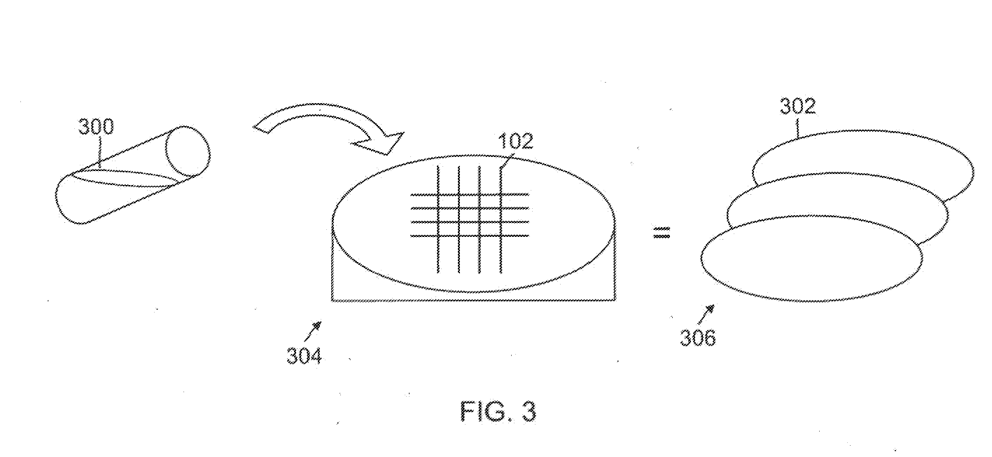

FIG. 4 shows a cross-section of another embodiment of the biological construct of the present invention;

FIG. 5 shows an embodiment of the biological construct as applied to a medical device; and

FIG. 6 shows an embodiment of the biological construct as applied to a hydrogel.

FIG. 7 shows a cross section of an embodiment of the layered polymeric biomatrix that mimics or corresponds with the three phases of the physiological healing process.

### DESCRIPTION OF THE PREFERRED EMBODIMENTS

The detailed description set forth below in connection with the appended drawings is intended as a description of presently-preferred embodiments of the invention and is not intended to represent the only forms in which the present invention may be constructed and/or utilized. The description sets forth the functions and the sequence of steps for constructing and operating the invention in connection with the illustrated embodiments. It is to be understood, however, that the same or equivalent functions and sequences may be accomplished by different embodiments that are also intended to be encompassed within the spirit and scope of the invention.

The present invention provides a biological construct and method for tissue remodeling and/or drug delivery following medical device implantation by utilizing a cyto-compatible, layered, bio-compatible polymeric biomatrix optimally constructed with a specialized surface texture of grain sizes up to 100 nm seeded with various therapeutic agents.

The biological construct may be used as an implantable device for controlled-release drug delivery and/or tissue regeneration system. The biological construct may be non-covalently or covalently layered with coatings of organic or semi-synthetic, nano-textured polymer. The nano-textured polymer may comprise pharmaceutical substances, such as growth factors, ligands, antibodies, and/or other beneficial biologically active agents for the purposes of controlled, differential substance/drug delivery into the luminal and abluminal surfaces of the tissue, and the attraction of target molecules/cells that will regenerate functional tissue and restore anatomic and physiologic integrity to the organ. The composition and construction of the polymer will be designed to facilitate the release of therapeutic agents in a temporal order that mimics the order of physiological processes that take place during natural organogenesis and tissue regeneration. This design (composition, thickness, elution kinetics, etc) can be modified to affect the specific regenerative properties of the implanted or injured tissue. The healing process may also be augmented by the addition of a tissue-specific, biologically engineered cell sheet 302, which may be overlaid onto the device along with its extracellular matrix. This may include endothelial progenitor cells, adult stem cells, embryonic stem cells, endogenous cardiac-committed stem cells, and other multipotent primitive cells capable of differentiation and restoring anatomic and physiologic integrity to the organ.

The biological construct comprises a polymeric compound designed with a nanophase surface texture, and various therapeutic agents, for the purpose of tissue regeneration and/or controlled delivery of growth factors and drugs after it is implanted into tissues, vessels, or luminal structures within the body. The invention may be applied to, but is not limited to any medical implant intended for vascular, cardiac, eye, bladder, cartilage, central and peripheral nervous system, lung, liver, pancreatic, stomach, smooth and skeletal muscle, visceral, renal, reproductive, epithelial and/or connective tissue application.

The following terms, as used herein, shall have the following meanings:

The term “delivery vehicle” refers to platforms, such as medical devices or medical substances that are introduced either temporarily or permanently into a mammal for the purposes of treating a disease, complication of a disease, or medical condition. This delivery vehicle can be introduced surgically, percutaneously, or subcutaneously into vessels, organs, cartilage, neural tissue, flesh, ducts and/or luminal structures within the body. Medical devices include, but are not limited to a stent, vascular graft, synthetic graft, valve, catheter, filter, clip, port, pacemaker, pacemaker lead, occluder, defibrillator, shunt, drain, clamp, probe, screw, nail, staple, laminar sheet, mesh, suture, chest tube, insert, or any device meant for therapeutic purposes. These devices may comprise titanium, titanium oxide, titanium alloy, stainless steel, nickel-titanium alloy (nitinol), cobalt-chromium alloy, magnesium alloy, carbon, carbon fiber, and/or any other biocompatible metal, alloy, or material. Medical substances include gels, such as hydrogels.

The term “nano-phase” or “nano-textured” are defined as having a surface texture with a grain size up to approximately 100 nanometers (nm). This includes, but is not limited to random or non-random patterns, which may include nano-spheres, nano-fibers, or nano-tubes.

The term “polymer” refers to when a molecule formed from the union of multiple (two or more) monomers. The polymer may be preferably amphipathic, and may be organic, semi-synthetic, or synthetic. Examples of polymers relevant to the present invention include, but are not limited to biologically tolerated and pharmaceutically acceptable poly(l-lactic acid) (“PLA”), poly(glycolic acid) (“PGA”), poly(lactic-co-glycolic acid) (“PLGA”), polycaprolactone (“PCL”), poly(ether urethane), Dacron, polytetrafluorurethane, polyurethane (“PU”), and/or silicon. The polymer may also include naturally occurring materials such as collagen I, collagen III, fibronectin, fibrin, laminin, cellulose ester, or elastin.

The term “nano polymer” or “nano-textured polymer” refers to the polymer (described above) with a naonophase surface roughness (grain size up to approximately 100 nm).

The term “therapeutic agent,” refers to any therapeutic substance or biological agent, or “beneficial biologically active agents” that is administered to the tissues or organs of a mammal to produce a beneficial effect. With respect to the present invention, therapeutic substances include antiproliforative agents, growth factors, antibiotics, thrombin inhibitors, immunosuppressive agents, antioxidants, peptides, proteins, lipids, enzymes, vasodilators, anti-neoplasties, anti-inflammatory agents, ligands (peptides or small molecule that binds a surface molecule on target cell), linker molecules, antibodies, and any janus kinase and signal transduction and activator of transcription (“JAK/STAT”) or AKT pathway activators are especially relevant. Biological agents include adult and/or embryonic stem cells, endogenous stem cells (e.g. endogenous cardiac-committed stem cells), and progenitor cells. These therapeutic agents are meant to be seeded into the polymeric material listed above.

The term “bioscaffold” refers to the polymeric backbone or lattice where therapeutic agents may be seeded. The bioscaffold may be biodegradeable (erodable) or non-biodegradeable (depending on the application) and can made from the polymeric mediums described above, insuring that that when implanted into the body, the polymer does not produce an adverse effect or rejection of the material. The architecture of the bioscaffold will attempt to mimic the native biological extracellular matrix of the tissue it is meant to regenerate. For example, the bioscaffold surface may contain weaves, struts, and coils.

The term “biomatrix” refers to the nano-textured biological construct, with or without a bioscaffold, and the therapeutic agent seeded within (drugs, living cells, etc).

The term “biodegradeable” refers to a material that can be broken down or eroded by chemical (pH, hydrolysis, enzymatic action) and/or physical processes once implanted into the body and exposed to the in-vivo physiological environment. The kinetics of this process can take from minutes to years. The subsequent components are non-toxic and excretable.

The term “cell sheet” refers to a specialized, tissue-specific population of cells grown on a scaffold. The sheets 302 are cultured ex-vivo 304 and subsequently harvested, along with their extra-cellular matrices, overlaid onto the nano-textured construct, and transplanted into host tissues to promote regeneration.

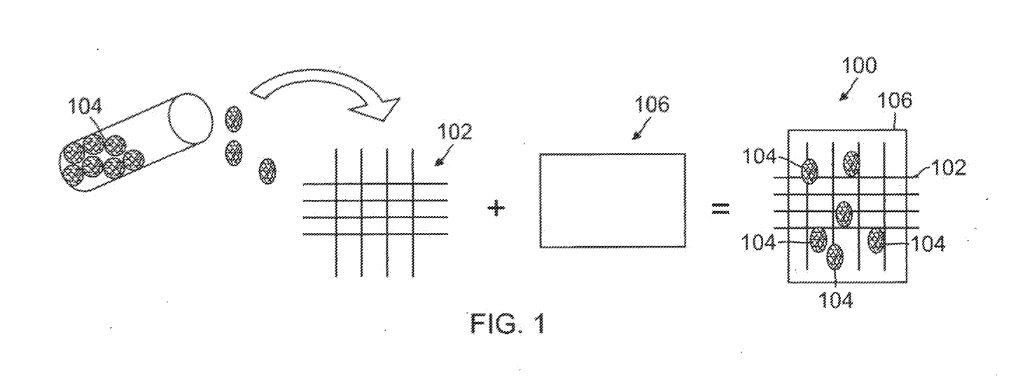

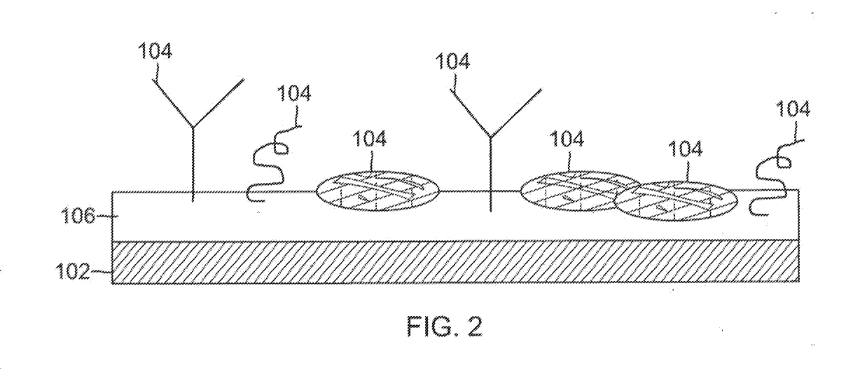

As illustrated in FIGS. 1 and 2, the nano-textured polymeric biomatrix 100 comprises an amphipathic organic, synthetic, or semi-synthetic polymeric material or bioscaffold 102 and the therapeutic agent 104 and/or 300 seeded within. The therapeutic agent 104 and/or 300 may be incorporated directly into a polymeric solution in a random or nonrandom fashion. The therapeutic agent 104 and/or 300 may be added directly, or the therapeutic agent 104 and/or 300 may be encapsulated, for example, enveloped into a microbubble, microsphere, or something of the kind before being added to the polymeric solution. The therapeutic agent 104 and/or 300 may be covalently or non-covalently coupled to the polymer. Depending on the chemical nature and molecular weight of the therapeutic agent 104 and/or 300, it may also be positioned between layers of polymers 102. The amount, concentration, or dosage of the therapeutic agent 104 and/or 300 seeded within the polymeric biomatrix 100 will be optimized for the target tissue and defined as the amount necessary to produce a therapeutic effect.

The nano-textured polymeric biomatrix 100 serves as a timed-release drug delivery system. After implantation, the construct is exposed to a physiological environment, and subsequently begins to erode and release at least one therapeutic substance 104. The erosion kinetics of the polymeric biomatrix 100 depends on the polymer density, choice of lipid membrane, glass transition temperature, and the molecular weight of the seeded substances and biological agents. In some embodiments, the biomatrix 100 may be comprised of different layers, types and densities of polymer, so that the erosion kinetics will be different throughout the construct. This will ensure healthy tissue regeneration (via the release of therapeutic substances) along with timed substance delivery (due to the degradation of the polymer) to maximize the biocompatibility of the implantable construct. The biological construct may be constructed such that the programmable nature of the device can be used for temporal, qualitative, and quantitative release of tissue-specific, therapeutic substances. The order, type, and dosage of substances eluted will be programmed to mimic the physiology that is observed in naturally occurring cellular environments during organogenesis, and/or tissue and/or organ regeneration during healing.

Thus, the nano-textured polymeric biomatrix 100 may be designed to facilitate controlled three-dimensional drug delivery and optimized to improve tissue regeneration. For example, the polymer 102 can serve to protect or preserve the biological agents 300, as they may not be exposed to the physiological environment until the polymeric portion of the biomatrix effectively erodes. In some embodiments, the polymeric portion may be in liquid or lyophilized phase at room temperature (approximately 25° C.) and subsequently change phase or conformation after implantation or direct injection at core body temperature (approximately 37° C.).

The nano-textured polymer 102 may also prepare the cellular environment by releasing buffers, inhibitors, or growth factors that will enhance the efficacy of a seeded therapeutic biological agent 300 or therapeutic substance 104 before it is released. This may also serve to protect the tissue from the acidity generated as a result of polymeric degradation.

In some embodiments, the constitution of the polymer may differ on different aspects of the construct. The surface of the polymeric bioscaffold 102 will be nano-textured to increase favorable cellular responses by optimizing surface chemistry, hydrophilicity, charge, topography, roughness, and energy. The surface of the polymeric bioscaffold 102 can be nano-textured 106 by methods described previously by Webster, et al. (5, 6, 14-18, 25, 26, U.S. patent application Ser. No. 10/793,721). Briefly, nano-textures may be generated with nanoparticles having grain sizes up to approximately 100 nm (carbon nano-tubules, helical rosette nano-tubes, nano-spheres, nano-fibers, etc). The nanoparticles may be transferred to the surface of a polymeric bioscaffold 102 comprising, for example, PLGA, PU, or the like, using specialty molds, hydrogel scaffolds, NaOH treatment, and sonication power. The surface roughness can be evaluated prior to implantation using scanning electron microscopy, if necessary. The nano-texture of each polymeric layer will not only improve the biocompatibility and cellular responses to the surface, but will also augment the bond between layers as well.

As shown in FIGS. 1 and 2, in some embodiments, a specialized population of tissue-specific cells 300 including, but not limited to, stem cells and progenitor cells, may be seeded withinin the polymeric bio-scaffold.

The nano-textured polymeric biomatrix 100 can be securely affixed to a delivery vehicle or a medical platform 400 by dipping, ultrasonic spray coating, painting, or syringe application. Dipping is a common method, and involves submerging the platform into a liquid solution (dissolved polymer) of the biomatrix. This can also be achieved by spraying the platform 400 with the liquid solution. The platform 400 can be dried and re-dipped or re-sprayed with different solutions to create specific, successive, biomatrix layers with independent functions. The multiple layers can also provide structural support for the construct and the polymeric density can be carefully controlled and altered to control elution kinetics. In addition, the concentration and combination of substances can be varied depending upon the polymeric thickness and/or number of layers in the polymer to control elution kinetics.

Select biological agents (antibodies, cells, etc.) may be covalently or non-covalently attached to the construct layers after it is dipped or sprayed. In some embodiments, the polymeric biomatrix 100 may not require a medical platform 400. Instead, it may be comprised of layers of biological agents and substances 306 with the layering providing the structural integrity.

In some embodiments, the biological construct for tissue regeneration in the present invention capitalizes on its likeness to natural architecture, nano-phase surface topography and the unique substance delivery system to improve the biocompatibility of the implantable construct 402 by attracting endothelial progenitor cells and other reconstructive factors, stimulating healing, and better reconstituting the injured tissue. Injured tissue includes any damage to tissue due to diseased conditions, disorders, and abnormalities, as well as any physical sustained injury, including those incurred during surgery.

The reconstructive sequence and coordination of the cellular events involved in physiological healing are well conserved among tissue types. The current invention capitalizes on this fact but also offers the opportunity to create tissue-specific implantable devices that are biocompatible and have the capability of delivering discrete regenerative factors and pharmaceutical substances (in a physiological fashion) that serve to enhance the reconstruction of a particular tissue or organ type.

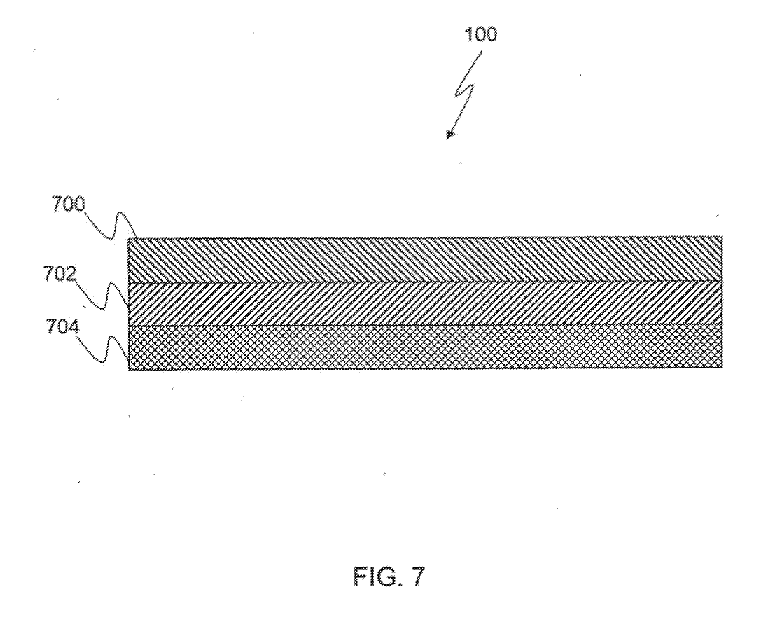

In some embodiments, the different phases of tissue regeneration (inflammatory, proliferative, and remodeling) can be represented in three, discrete polymeric layers of the construct, each layer seeded with appropriate therapeutic agents or regenerative factors to aid the healing process in a particular phase of remodeling as illustrated in FIG. 7. The outermost layer contains factors corresponding to the inflammatory phase, the middle layer is designed to enhance cellular migration and differentiation in the proliferative phase, and the innermost layer serves to provide trophic factors to support cellular function, signaling, and survival within the remodeling phase. These layers can also be sub-stratified or sublayered to further direct cellular-signaling within the environment and control the release of substances. Polymeric variety, composition, and thickness can be altered to control the degradation rates of the construct such that rates of substance release match the temporal scale of the physiological healing process (described below).

The specific type(s) and density of polymeric material will vary with respect to the type of tissue it is meant to reconstruct. Naturally derived materials such as collagen, hyaluronan, fibrin, chitosan, and gelatin are not only useful in soft tissue, dermal, vascular, skin, cartilage, and bone repair and engineering, but also possess an innate ability to facilitate cellular communication, differentiation, growth patterning, and the control of vascular sprouting, making them excellent candidates for the “inflammatory” and “proliferative” layers of the construct. Synthetic polymers such as PGA, PLA, PLGA, PCL, PU, and PEG are highly elastic, demonstrate a wide range of biodegradability rates, and have been shown to augment musculoskeletal, fibrovascular, skin, bone, and cartilage remodeling. These materials are durable enough to support the “remodeling” layers of the implant. Additionally, because organic and synthetic polymers demonstrate distinct strengths in tissue healing applications, polymeric blends are emerging as promising vehicles of controlled drug delivery. The blends offer major advantages in tissue reconstruction in that, through manipulation of the relative molecular masses and ratios, they allow for more careful rates of degradation while improving the biocompatibility of the implant. Adjusting the polymer:polymer blend ratio provides an additional level of control over the substance delivery from the device because the materials can be designed to be more or less sensitive to environmental factors like pH, temperature, enzymatic activity, and water. Additionally, there are now mathematical models to predict degradation and drug elution rates from polymeric mixtures, which will prove useful for applications in different tissue types. For example, PLGA/Poly-L-Lactide (“PLLA”) co-polymers (with molecular masses ranging from 4,400; 11,000; 28,000; and 64,000 Daltons) with 50:50 lactic acid to glycolic acid ratio produce polymers with degradation rates ranging from weeks to months. These polymers are commercially manufactured, available for purchase (Alkermes), and can even be sold as individual therapeutic particles comprised of PLGA encapsulated substances (Pfizer, Novartis, Johnson & Johnson, etc.). Taking into account the wide application of these polymeric mixtures, each layer of the current invention may be comprised of a different ratio of organic:synthetic polymers, depending on the desired function of the layer (inflammatory, proliferative, or remodeling).

In all corporeal tissues, the healing process consists of a carefully coordinated phases of biochemical and metabolic events necessary to remodel the injured tissue. While the cellular interactions within these phases can often overlap and even coincide, the sequence of events has been carefully considered in developing the current invention. This sequence includes three phases: the inflammatory phase, the proliferative phase, and the remodeling phase.

The inflammatory phase is characterized by the removal (phagocytization) of bacteria and cellular debris from the site of injury and the preliminary deposition of protein to provide interim structural support to the site of implantation/injury. Immediately following insult, inflammatory factors (cytokines, histamine, leukotaxin, necrosin, bradykinin, prostaglandins, prostacyclins, thromboxane) and glycoproteins are secreted. Together, these factors effect a brief period of vasoconstriction (thromboxane and prostaglandins) to prevent further bleeding, followed by prolonged vasodilation (histamine) to facilitate the entry of leukocytes (T-cells) and monocytes to the wound site. During this time, fibrin, fibronectin, hyaluronan, glycosoaminoglycans, and proteoglycans bind and cross-link to create a preliminary extracellular matrix, or scab, that not only serves to support the tissue until collagen is deposited, but also as a mesh to facilitate the mobility and migration of other reconstructive cells. Matrix formation is coordinated temporally and spatially by the up-regulation of matricellular proteins (galectins, osteopontin, SPARC, thrombospondins, tenascins, vitronectin, and CCN proteins), which signal cellular interactions. Fibronectin, neuropeptides, and growth factors (TGF-β, Bone Morphogenic Proteins (“BMPs”), such as BMP-4, Insulin-Like Growth Factor (“IGF”), VEGF, FGF, Platelet Derived Growth Factor (“PDGF”)) attract polymorphonuclear neutrophils which dominate the area and clean the wound of debris and bacteria (via phagocytosis and protease activity) for approximately 3 days. Following this period (days 3 and 4), monocytes mature into macrophages, (replacing neutrophils), and resume the job of clearing the injured area of debris and preparing it for the next phase of healing. In response to the low oxygen environment, macrophages also release tissue-specific factors that stimulate angiogenesis, induce the creation of a permanent extra cellular matrix, and mobilize progenitors and/or cell-cycle activators that will stimulate cellular regeneration during the proliferative phase.

As the numbers of macrophages and inflammatory factors are reduced and the numbers of fibroblasts increase, the healing process transitions from the inflammatory to the proliferative phase. During this phase, several cellular events overlap to stimulate new tissue growth: angiogenesis, the formation of granular tissue, fibroplasia, epithelialization, and contraction.

Angiogeneis is stimulated by the migration of fibroblasts and endothelial cells to the injury. These cells push through the extracellular matrices of healthy issue and migrate to the injury to provide oxygen and nutrients; subsequently, new vessels are formed. While endothelial cells are attracted to the wound site chemotactactically (by the preliminary mesh created by fibrin and fibronectin) growth and proliferation of these cells is stimulated by the lack of oxygen and acidity of the environment. As they multiply, the scar becomes re-perfused and re-oxygenized, and the number of endothelial cells is reduced.

One week following injury, fibroblasts become the main cell type present in the wounded area. The goal of these cells is to recreate the structural integrity of the insult by laying down granulation tissue (new vessels, fibroblasts, inflammatory factors, endothelial cells and the provisional extracellular matrix) and collagen. Fibroblasts are stimulated by growth factors and matricellular proteins to deposit fibronectin, glycoproteins, glycosoaminoglycans, proteoglycans, elastin, and collagen. These substances work together to create the new extracellular matrix. Collagen deposition persists for up to four weeks and will ultimately account for closing the wound and providing the stability of the new matrix. As it reaches maturity, the fibroblasts undergo apoptosis, evolving the scar from a cell-rich, preliminary structure, to a fortified collagen scaffold. Epithelial cells begin to proliferate and migrate from the wound edges across this matrix (under the scab) to resurface the injury. Once covered, the native tissue cells replicate to create healthy tissue.

After the structural and nutritive support has been returned to the site of injury, it begins to contract. This process can last for several weeks and is caused by newly differentiated myofibroblasts, which are similar to smooth muscle cells. The actin component in the myofibroblasts actively pulls the extracellular matrix edges together to close the wound and break down the preliminary matrix.

This signals the end of fibroblast proliferation and migration, as well as the beginning of the remodeling phase. When the rates of collagen synthesis match those of collagen degradation, the newly created tissue begins to mature. Collagen fibers re-arrange and align to remodel the cellular composition and reinstate the strength of the tissue. This process can last from months to a year, until the wound is properly healed.

To improve drug delivery, wound healing, and tissue remodeling, the biological construct of the present invention comprises biocompatible polymeric layers, wherein each layer comprises a nanophase surface texture to improve the biocompatibility of the biological construct, and a therapeutic agent seeded within the biocompatible polymer. The therapeutic agent in each biocompatible polymeric layer corresponds with a different stage of wound healing and tissue remodeling.

For example, a first layer 700 may comprise at least one inflammatory response agent that corresponds to the inflammatory phase of wound healing and tissue remodeling. Thus, inflammatory response agents may be therapeutic agents involved in the removal of bacteria and cellular debris as well as the depositing of proteins to provide preliminary or interim structural support or extra cellular matrices to further facilitate phagocytization, create the structural framework for healing and tissue remodeling, and transition to the next phase of healing and tissue remodeling. Examples of inflammatory response agents include neural mitogens, cell migration activators, thrombin activators, differentiation agents, growth factors, and trophic factors.

A second layer 702 may comprise at least one proliferative agent corresponding to the proliferative phase. Proliferative agents include therapeutic agents that may induce replication and facilitate angiogenesis, the formation of granular tissue, fibroplasias, epithelialization, and contraction. Examples of proliferative agents include replication inducing agents, stem cell mobilizing factors, endothelial cell attractants, neural mitogens, cell migration activators, differentiation agents, angiogenic agents, growth factors, trophic factors, neuroprotective agents, cell-cycle activators, extracellular matrix forming agents, and neurite outgrowth agents.

A third layer 704 may comprise at least one remodeling agent corresponding to the remodeling phase. Remodeling agents include therapeutic agents that may be involved in the differentiation, maturation, re-arrangement, and strengthening of cells and tissues. Examples of remodeling agents include stem cell mobilizing factors, endothelial cell attractants, cell-cycle activators, neuroprotective agents, and anti-scarring agents.

Since wound healing progresses from the inflammatory phase to the proliferative phase to the remodeling phase, in the preferred embodiment the first layer 700 containing the inflammatory response agent is the outer-most layer of the layered biological construct, the second layer 702 containing the proliferative agent is the next inner layer of the layered biological construct, and the third layer 704 containing the remodeling agent makes up the inner-most layer of the biological construct. This controls the delivery of the appropriate therapeutic agent at the appropriate time, specifically, release of inflammatory response agents during the inflammatory phase, release of the proliferative agents, during the proliferative phase, and release of the remodeling agents, during the remodeling phase.

As the inflammatory phase, the proliferative phase, and the remodeling phase overlap, the inflammatory response agents, the proliferative agents, and the remodeling agents may also overlap. In other words, therapeutic agents used in any one layer may also be suitable and present in another layer.

Furthermore, the biocompatible polymer of the first layer 700 and second layer 702 may comprise naturally-derived polymers such as collagen, hyaluronan, fibrin, chitosan, and gelatin because of their innate ability to facilitate cellular communication, differentiation, growth patterning, and the control of vascular sprouting. The biocompatible polymer of the third layer 704 may comprise synthetic polymers such as PGA, PLA, PLGA, PCL, PU, and PEG. In some embodiments, biocompatible polymer of the first, second, and/or third layer 700, 702, 704 may comprise a blend of naturally-derived polymers and synthetic polymers.

Detailed iterations of this embodiment are listed below:

1. The Pancreas.

Diabetes Mellitus is a pandemic disease affecting millions world-wide. The pathogenesis of diabetes results from the destruction of pancreatic B-cells, defective insulin action, or both. A better understanding of the regeneration both exocrine and endocrine pancreatic tissue will provide much needed, and improved therapies for these patients.

Cellular turnover in the mature pancreas is guided by local signaling mechanisms within Notch pathway intermediates, otherwise known as “binary fate choice.” This pathway is a key determinant of whether a pancreatic progenitor cell will proliferate (remain in the cell-cycle) or differentiate (exit the cell cycle to obtain its cellular identity). Pancreatic endocrine and exocrine cells are regenerated from a population of stem cells, called pancreatic progenitor cells (Pdx1+), located in the pancreatic ducts. Pdx1+ cells receiving the Notch signal, will repress genes specific to differentiation. This promotes progenitor cell renewal (mitogenesis) and discourages cellular differentiation. Pdx1+ cells that do not receive the Notch signal undergo the up-regulation of the transcription factor Neurogenin 3 (Ngn3) and differentiate into mature endocrine (B) cells. Glucagon-like peptide-1 (GLP-1) has also been shown to induce β-cell replication in adult tissue.

Considering this, one iteration of the current invention could be constructed with substances known to induce replication seeded into the “proliferative” layer of the device, namely Ngn3 and GLP-1, along with a careful combination of growth factors and therapeutic agents and matricellular signaling proteins, with the goal of not only healing the tissue, but also stimulating pancreatic progenitor differentiation (i.e. suppressing the Notch signal) without disturbing the proliferation of the stem cell population.

2. The Heart.

Together, coronary heart disease, cardiomyopathy, cardio-vascular disease, and ischemic heart disease comprise the leading causes of death in the United States. In addition to congenital heart defects, acquired injuries to the myocardial tissue and its associated vasculature create grave, and often, deadly complications for both adolescent and adult patients. The cells of the myocardium, or heart tissue, are the unitary elements that can improve or define cardiac disease. Thus, improving the restorative potential of these cells and the overall tissue is of critical importance.

The mature heart is comprised of several different cell types (cardiac muscle, smooth muscle, the conduction system, endothelial cells, valvular cells, and interstitial mesenchymal fibroblast cells), all of which are important for effective structural and functional formation. The pre-natal heart is the first organ to form in vertebrates, but once mature, the post-natal heart is a classically non-regenerative organ, that is cardiac muscle cells do not have the ability to regenerate. Considering this, along with the instance of cardiac disease, there is an increasing amount of research going into cell-cycle activation and cellular transplantation to restore the diseased heart.

The most promising work toward cardiac reconstruction has focused on potential sources of adult cardiac precursors and progenitor cells for transplantation. There are several candidates, namely hematopoietic stem cells, mesenchymal stem cells, and endothelial progenitor cells. In order for successful cardiac restoration, these cells must demonstrate the ability to differentiate, self-renew, integrate and communicate with resident cells, and exhibit appropriate electrical coupling. While the exact mechanism that governs the differentiation of these progenitors is still somewhat of an enigma, there are several candidates that have demonstrated the ability to develop into mature cardiomyocytes, smooth muscle cells and/or endothelial cells: ckit1+ cells, Sca1+ cells, Sca1+ (ABCG2) cells, cardiospheres, and Isl1+ cells. The results of these studies have been mixed, but encouraging. Some have demonstrated that stem cell therapy is still not fully competent at regenerating cardiac muscle, it has secondary effects, improving cardiac function by promoting angiogenesis and cell survival through cardio-protective mechanisms.

Taking all of this into account, another iteration of the current invention could seed stem cell mobilizing factors [G-CSF], endothelial cell attractants [GM-CSF, CSF-1, G-CSF, M-CSF, c-mpl ligand (MGDF or TPO), erythropoietin (EPO), stem cell factor (SCF), flt3 ligand, vascular endothelial growth factor (VEGF), fibroblast growth factor (FGF)-3, FGF-4, FGF-5, FGF-6, FGF-7, FGF-8, FGF-9, basic fibroblast growth factor, platelet-induced growth factor, transforming growth factor beta 1, acidic fibroblast growth factor, osteonectin, angiopoietin 1, angiopoietin 2, insulin-like growth factor, the antibody or antibody fragment has a binding affinity to one or more of the following: CD34 receptors, CD133 receptors, CDw90 receptors, CD117 receptors, HLA-DR, VEGFR-1, VEGFR-2, Muc-18 (CD146), CD130, stem cell antigen (Sca-1), stem cell factor 1 (SCF/c-Kit ligand), Tie-2, HAD-DR] and cell-cycle activators [thymosin β-4, Homeobox Protein Nkx-2.5 (“Nkx2.5”), SV40 Large T-antigen, D-Type Cyclins, Cyclin-Dependent Kinase 2 (“CDK2”), dominant interfacing TSC2, p193, p53, and p38, Mitogen-Activated Protein Kinase (“MAPK”), Cyclin A2, BCK2 Gene, also called bypass of kinase C protein (“Bck-2”), GLP-1, and Insulin-Like Growth Factor-1 (“IGF-1”)], into the “proliferative” and “remodeling” polymeric layers to mobilize cells, attract reparative cells and encourage differentiation following stem cell delivery. Providing a balance of substances and growth factors that would augment the benefits of stem cell therapy, up-regulate the cell-cycle, promote angiogenesis, and/or facilitate cell-to-cell signaling between resident cells and stem cells within a biocompatible polymer (executed in a physiological manner) would undoubtedly improve the chances for successful cardiac function and restoration.

3. The Central Nervous System.

Neural cell survival and regeneration is a hugely important issue with respect to brain and spinal cord injury, aging, and diseases of the central nervous system. Huntington's, Parkinson's, and Alzheimer's disease all result from neural cell degeneration, or death and are affecting millions. Further, acute injury such as stroke can have devastating consequences on motor and cognitive function. Neuronal stem cells (NSCs) have demonstrated the ability to divide and differentiate in developing and mature brain tissue, however regeneration in the adult brain has proven to be a much more complicated issue. Neural tissue has a unique response to injury, and NSCs (transplanted or endogenous) require selective neurotrophic factors for successful cellular signaling and survival. The delivery of bio-agents and neurotrophic factors can be complicated by the blood brain barrier and is further limited by insufficient and inappropriate delivery techniques. Biocompatible polymers and hydrogels (PEG and PLGA) have shown promise in effective substance delivery as they retain the bioactivity of the neurotrophins and biologically active substances and have easily controllable rates of degradation.

Considering this, another iteration of the current invention proposes to create a layered polymeric structure that will have the ability to execute carefully controlled extrinsic cues that direct cellular differentiation, prevent apoptosis, promote myelination, and support long-term cell survival in regenerating neural tissue, while preventing glial scarring (that can inhibit axonal growth). The “inflammatory” and “proliferative” layers will be seeded with neural mitogens (Epidermal Growth Factor (“EGF”), basic fibroblast growth factor (“bFGF”), large T-antigen, GM-CSF) to stimulate differentiation, mitosis, and cellular migration. The lower strata of the “proliferative” layer will be filled with trophic factors, such as substances that promote cell survival, astrocyte and oligodendrocyte growth, and neuroprotective agents that promote long-term neuro-protection: PDGF, FGF2, Bone Derived Neurotrophic Factor (“BDNF”), Neurotrophin 3 (“NT3”), Neurotrophin 4/5 (“NT4/5”), Nerve Growth Factor (“NGF”), and bFGF, Glial Derived Neurotrophic Factor (“GDNF”), Connective Tissue Nutrient Formula (“CTNF”), Thyroid hormone T3, and galectin. Finally, the “remodeling” layer of the construct will contain substances (chromaffin cells) known to enhance long-term neural cell survival.

4. Skin.

Skin is a dramatically complex and multi-purpose organ. Comprising one tenth of the body's mass, the cellular makeup of this organ is diverse, thus attempts to improve skin regeneration and healing must take into consideration a construct that can facilitate a variety of functions and cellular processes. The skin as an organ is stratified, so the healing device must be designed to address wounds of various thickness, encourage angiogenesis, provide a robust barrier, be non-toxic, and anti-necrotic, all while minimizing pain, inflammation, and scarring.

The current invention has the capability to do so, as the basis for the construction of the device is polymeric layering, such that each layer can support a different function. In the iteration of the skin, the polymeric layers would be comprised of naturally occurring substances such as collagen, fibronectin, polypolypeptides, hydroxyapetites, hyaluronan, glycosylaminoglycans, chitosan, or alginates. These organic polymers can be blended with other biocompatible, synthetic polymers to better control elution and degredation kinetics as well.

In this iteration, the superficial, or “inflammatory” layer would be seeded with PDGF, cytokines, and compliment pathway activators to improve neutrophil migration, host defenses against infection, and thrombin activation. The second or “proliferative” layer will be augmented with integrins, TGF-α and TGF-β (the specific factor will vary depending on the age of the patient), IGF-1, BMPs, EGF, bFGF, VEGF, Ang-1, and Hepatic Growth Factor/Scatter Factor (“HGF/SF”) to induce differentiation, cellular migration, angiogenesis, and encourage cell-survival. The final layer, the “remodeling” layer will be constructed to minimize the scarring process. It will lack TGF-β, and PDGF as these factors promote scarring in mature tissue. Instead, this layer will contain a collagen bioscaffold and matricelluar proteins to encourage healthy cell-to-cell signaling and physically support the newly remodeled tissue. The thickness of these polymeric layers will ultimately depend on the relative depth of the wound.

5. The Retina.

Ocular remodeling has posed a huge challenge in the field of regenerative medicine due to the fact that the tissues of the eye are not only functionally distinct, but also arise from different populations of emybryonic tissue. While this issue is far from being solved, there have been advances with respect to retinal regeneration. Recent studies have found that human embryonic stem cells (ESC's) can be directed to a retinal cell fate using specific inhibitors and growth factors.

With this in mind, the current invention could be combined with stem cell therapies to provide micro-environmental cues and improve the success of the current strategies following stem cell implantation. The outer, “inflammatory” layer of the polymer would contain BMP and Wnt inhibitors and IGF-1 to coordinate appropriate differentiaton. The second, “proliferative” layer will include NGF, NT3, NT4/5, FGF-2, GDNF to activate the cell cycle, facilitate cellular migration, and modulate the formation of the extra cellular matrix. Neurotrophin 4 (“NT4”), BDNF, and Connective Tissue Nutrient Formula (“CTNF”) will be added to encourage neurite outgrowth. The final layer will include Tumor Necrosis Factor-α (“TNF-α”) to provide neuro-protection to support the integrity and function of the newly differentiated neurons and glia.

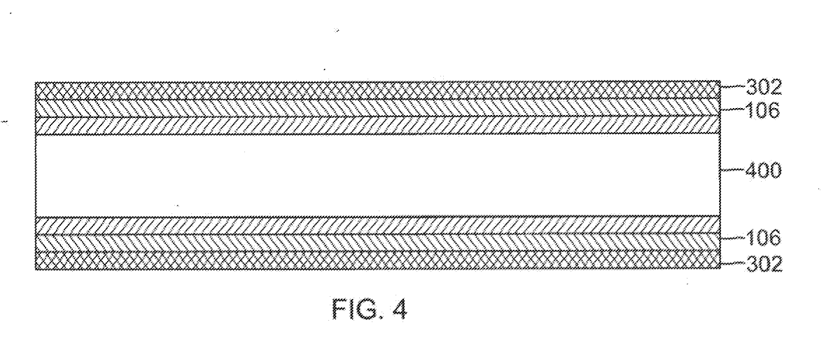

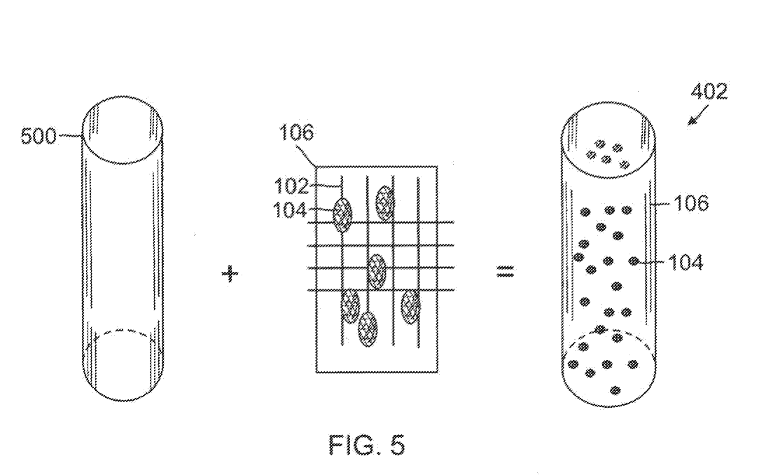

As shown in FIGS. 4-5, in some embodiments, the current invention will also provide a method for addressing the problem of re-stenosis and late thrombosis following endovascular or endoluminal device placement by implanting a biological construct (polymer or polymer+platform) whose nano-surface features and polymeric constitution may enhance endothelial healing, mitigate smooth muscle vascular cell adhesion, and ultimately promote vascular reconstitution in patients suffering from cardiovascular disease. The nano-textured, polymeric biomatrix 100 can be formulated and applied (sprayed, dipped, painted) onto a device 500, such as a stent, vascular graft, valve, catheter, filter, clip, port, pacemaker, pacemaker lead, defibrillator, shunt, or any endovascular or endoluminal device designed to treat the complications associated with vascular disease. In this instance, the construct would seek to emphasize the method of endothelial healing facilitated by the nano-phase texture of the polymer and the platform of the device 500. The pattern of this nano-texture may be random and/or non-random; designed to effect the flow of blood, such as to facilitate the capture of endothelial progenitor cells; maximize lumen size; and minimize smooth muscle cell adhesion. The polymer 102 facilitates the controlled release of pharmaceutical compound 104 to abluminal and luminal surfaces of the construct. To facilitate controlled release the biomatrix 100 may contain layers of ligands, antibodies, and growth factors designed to bind and/or attract specific membrane molecules on target cells (endothelial progenitor cells), with the goal of augmenting endothelial healing. In this embodiment, these may include one or more of the following: anti-proliferative agents (paclitaxel, sirolimus, etc.), endothelial progenitor cells, endogenous cardiac-committed stem cells, Flk1+ progenitors, cardiosphere daughter cells, endothelial cell growth factors granulocyte macrophage colony-stimulating factor (“GM-CSF”, CSF-1), granulocyte colony-stimulating factor (“G-CSF”), macrophage colony-stimulating factor (“M-CSF”), erythropoietin, stem cell factor, vascular endothelial growth factor (“VEGF”), fibroblast growth factors (“FGF”) such as FGF-3, FGF-4, FGF-5, FGF-6, FGF-7, FGF-8, and FGF-9, basic fibroblast growth factor, platelet-induced growth factor, transforming growth factor beta-1, acidic fibroblast growth factor, osteonectin, angiopoetin-1, angiopoetin-2, insulin-like growth factor), smooth muscle cell growth inhibitors, antibiotics, thrombin inhibitors, immunosuppressive agents, antioxidants, peptides, proteins, growth factor agonists, vasodilators, anti-platelet aggregation agents, collagen synthesis inhibitors, extracellular matrix components, fms-like tyrosine kinase receptor-3 (“flt3”) ligand, c-mpl ligand, megakaryocyte growth and differentiation factor (“MGDF”) or thrombopoietin (“TPO”), ricin ligands, or any antibody or antibody fragment that has the binding affinity to one of the following: CD34 receptors, CD133 receptors, CDw90 receptors, CD117 receptors, HLA-DR, Flk1, VEGFR-1, VEGFR-2, Muc-18 (CD146), CD 130, stem cell antigen (Sca-1), stem cell factor (SCF/c-kit ligand), Tie-2, and/or HAD-DR. Together, this nano-textured device will promote endothelial healing and vascular reconstruction.

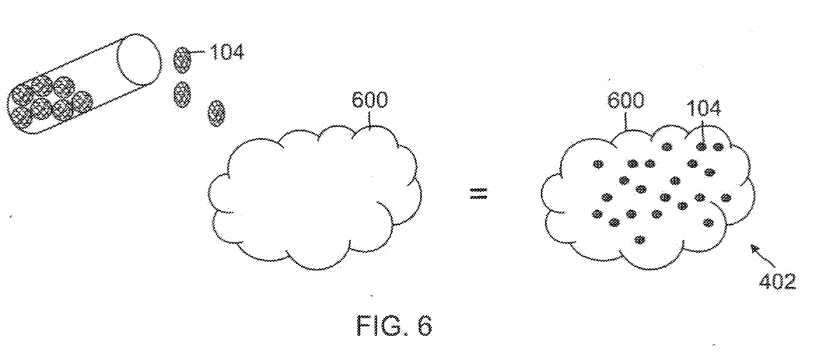

In another embodiment, the current invention provides a method for addressing the problem of cellular migration and survival following various forms of cell therapy. Therapeutic substances and biologically beneficial agents 104 and 300, respectively, can be applied directly to a specific lesion or insult in the tissue through the use of a nano-textured hydrogel 600 seeded with therapeutic agents 104 and/or 300 as shown in FIG. 6. Using minimally invasive surgical techniques to apply the gel 600, or “bio-dots,” the use of this polymeric medium can ensure proper placement and security of the cells, discourage cellular migration, improve cellular response, survival, and integration, and protect protein based substances seeded within. Additionally, the elution kinetics of the construct can be controlled by the rate of polymeric degradation, making the “bio-dots” inherently programmable.

In another embodiment, the current invention provides a method for addressing the problem of cellular rejection, migration, and partial thrombosis of the hepatic vasculature following islet transplantation procedures in insulin-dependent diabetic patients. Type I and late stage type II diabetics have impaired insulin and glucagon function, which compromises their endogenous ability to maintain euglycemia. In attempting to manage blood glucose levels, most patients undergo rigorous insulin replacement therapy in the form of subcutaneous insulin administration. While there have been advances in glycemic monitoring devices and insulin delivery systems, insulin therapy is still flawed; it is unable to mimic physiological insulin secretion, making patients extremely vulnerable to complications, primarily hypoglycemia. In an attempt to mitigate these complications, and to free patients of insulin dependency, experimental islet transplantation has become an option. As with many transplantations, this procedure is accompanied by the risk of partial thrombosis in the portal vein (and other small intra-hepatic vessels), islet cell rejection, poor cellular survival and function, and cellular migration. Additionally, anti-rejection drugs (immuno-suppressants) given after transplantation make patients vulnerable to opportunistic infection and have been shown to impair normal islet function. By seeding the islets 300 in a nano-textured polymeric bioscaffold 102, 106, the islets will be carefully deposited along with their extra-cellular matrices and growth factors through the portal vein into the hepatic host tissue. The polymer 102 will provide a stable, therapeutic environment for the islets, which will bio-mimic physiological conditions and encourage proper function. The ultimate goal of this application is to stimulate integration, and ultimately improve overall insulin and glucagon secretion. Functional islets can liberate diabetic patients from insulin dependency or reduce insulin dependency and allow them to realize the benefits of true glycemic control.

The nanophase surface properties of the construct will favor positive tissue remodeling following implantation through controlled drug delivery, optimized cyto-compatible surface characteristics, and favorable protein adsorption and cellular interaction. The application of the present invention may extend to, but is not limited to biological constructs in vascular, cardiac, epithelial, eye, bladder, cartilage, central and peripheral nervous system, lung, liver, pancreatic, stomach, smooth and skeletal muscle, visceral, renal, reproductive, and connective tissues.

While the current invention is unique compared to previous developments in the field, it seeks to emphasize the improved biocompatibility of the device, the controlled, physiological, substance elution system, and the nanophase surface features of the polymer.

The foregoing description of the preferred embodiment of the invention has been presented for the purposes of illustration and description. It is not intended to be exhaustive or to limit the invention to the precise form disclosed. Many modifications and variations are possible in light of the above teaching. It is intended that the scope of the invention not be limited by this detailed description, but by the claims and the equivalents to the claims appended hereto.

What is claimed is:

1. A biological construct for improved drug delivery and tissue remodeling, comprising:
a polymeric biomatrix, comprising:
a. a plurality of biocompatible polymeric layers, each biocompatible polymeric layer, comprising:
i. a nanophase surface texture comprising surface crystal grain sizes of less than or equal to approximately 100 nm arranged in a pre-determined pattern designed for improved, programmable, sequential drug delivery and specific tissue remodeling that is designed to recapitulate the natural healing process; and
ii. a therapeutic agent seeded within the biocompatible polymeric layer, wherein the therapeutic agent in the biocompatible polymeric layer corresponds with a phase of wound healing and tissue remodeling, wherein
b. the polymeric biomatrix, comprises:
i. a first layer comprising an inflammatory response agent, wherein the inflammatory response agent in the first layer is selected from the group consisting of a neural mitogen, a cell migration activator, a thrombin activator, a differentiation agent, a growth factor, and a trophic factor;
ii. a second layer comprising a proliferative agent, wherein the proliferative agent in the second layer is selected from the group consisting of a replication inducing agent, a stem cell mobilizing factor, an endothelial cell attractant, and a second neural mitogen, a second cell migration activator, a second differentiation agent, an angiogenic agent, a second growth factor, a second trophic factor, a neuroprotective agent, a cell-cycle activator, an extracellular matrix forming agent, and a neurite outgrowth agent; and
iii. a third layer comprising a remodeling agent, wherein the remodeling agent in the third layer is selected from the group consisting of a second stem cell mobilizing factor, a second endothelial cell attractant, a second cell-cycle activator, a second neuroprotective agent, and an anti-scarring agent, wherein
iv. each biocompatible polymeric layer comprises a sub-layer comprising a second therapeutic agent; and wherein
c. the biocompatible polymer is selected from the group consisting of poly(l-lactic acid) (“PLA”), poly(glycolic acid) (“PGA”), poly(lactic-co-glycolic acid) (“PLGA”), polyethylene glycol (“PEG”), polycaprolactone (“PCL”), poly (N-isopropylacrilamide) (“PIPAAm”), poly(ether urethane), dacron, polytetrafluorurethane, polyurethane (“PU”), silicon, cellulose ester, collagen I, collagen III, elastin, fibronectin, fibrin, fibrinogen, laminin, hydroxyapetites, hyaluronan, glycosylaminoglycans, chitosan, or alginates.

2. A biological construct for improved drug delivery and tissue remodeling to mimic natural healing of an injured tissue, comprising: a polymeric biomatrix, comprising a plurality of biocompatible polymeric layers, each biocompatible polymeric layer, comprising:
a. a nanophase surface texture comprising surface crystal grain sizes of less than or equal to approximately 100 nm arranged in a pre-determined pattern designed for improved, programmable, sequential drug delivery and specific tissue remodeling that is designed to recapitulate the natural healing process; and
b. a therapeutic agent seeded within the biocompatible polymeric layer, wherein the therapeutic agent in the biocompatible polymeric layer corresponds with a phase of wound healing and tissue remodeling;
wherein a first layer comprises an inflammatory response agent; a second layer comprises a proliferative agent; and a third layer comprises a remodeling agent.

3. The biological construct of claim 2, wherein the inflammatory response agent in the first layer is selected from the group consisting of a neural mitogen, a cell migration activator, a thrombin activator, a differentiation agent, a growth factor, and a trophic factor.

4. The biological construct of claim 2, wherein the inflammatory response agent is selected from the group consisting of epidermal growth factor (“EGF”), basic fibroblast growth factor (“bFGF”), large T-antigen, granulocyte macrophage colony-stimulating factor (“GM-CSF”), platelet derived growth factor (“PDGF”), cytokines, bone morphogenic proteins (“BMP”) inhibitors, Wnt inhibitors, and insulin-like growth factor-1 (“IGF-1”).

5. The biological construct of claim 2, wherein the proliferative agent in the second layer is selected from the group consisting of a replication inducing agent, a stem cell mobilizing factor, an endothelial cell attractant, a neural mitogen, a cell migration activator, a differentiation agent, an angiogenic agent, a growth factor, a trophic factor, neuroprotective agents, a cell-cycle activator, an extracellular matrix forming agent, and a neurite outgrowth agent.

6. The biological construct of claim 2, wherein the proliferative agent is selected from the group consisting of neurogenin (“Ngn3”), glucagon like peptide (“GLP-1”), granulocyte colony-stimulating factor (“G-CSF”), colony-stimulating factor (“CSF”), CSF-1, macrophage colony-stimulating factor (“M-CSF”), c-mpl ligand, megakaryocyte growth and differentiation factor (“MGDF”), erythropoietin (“EPO”), stem cell factor (“SCF”), fms-like tyrosine kinase receptor-3 (“flt3”) ligand, vascular endothelial growth factor (“VEGF”), fibroblast growth factor (“FGF”)-3, FGF-4, FGF-5, FGF-6, FGF-7, FGF-8, FGF-9, basic fibroblast growth factor (“bFGF”), platelet-induced growth factor, transforming growth factor (“TGF”) beta 1, acidic fibroblast growth factor, osteonectin, angiopoietin 1, angiopoietin 2, insulin-like growth factor, an antibody or antibody fragment, thymosin β-4, homeobox protein Nkx-2.5 (“Nkx2.5”), SV40 large T-antigen, D-type cyclins, cyclin-dependent kinase 2 (“CDK2”), dominant interfacing TSC2, p193, p53, and p38, mitogen-activated protein kinase (“MAPK”), cyclin A2, bypass of kinase C protein (“bck-2”), IGF-1, EGF, large T-antigen, GM-CSF, PDGF, FGF-2, bone derived neurotrophic factor (“BDNF”), neurotrophin 3 (“NT3”), neurotrophin 4/5 (“NT4/5”), nerve growth factor (“NGF”), glial derived neurotrophic factor (“GDNF”), thyroid hormone T3, galectin, integrins, TGF-α, TGF-β, BMPs, Ang-1, hepatic growth factor/scatter factor (“HGF/SF”), neurotrophin 4 (“NT4”), and connective tissue nutrient formula (“CTNF”).

7. The biological construct of claim 6, wherein the antibody or antibody fragment has a binding affinity to one or more of an antigen selected from the group consisting of CD34 receptors, CD133 receptors, CDw90 receptors, CD117 receptors, HLA-DR, VEGFR-1, VEGFR-2, Muc-18 (CD146), CD130, stem cell antigen (Sca-1), stem cell factor 1 (SCF/c-Kit ligand), Tie-2, and HAD-DR.

8. The biological construct of claim 2, wherein the remodeling agent in the third layer is selected from the group consisting of a stem cell mobilizing factor, an endothelial cell attractant, a cell-cycle activator, a neuroprotective agent, and an anti-scarring agent.

9. The biological construct of claim 2, wherein the remodeling agent is selected from the group consisting of colony-stimulating factor (“CSF”), CSF-1, G-CSF, M-CSF, c-mpl ligand, megakaryocyte growth and differentiation factor (“MGDF”), thrombopoietin (“TPO”), erythropoietin (“EPO”), stem cell factor (“SCF”), flt3 ligand, vascular endothelial growth factor (“VEGF”), fibroblast growth factor (“FGF”)-3, FGF-4, FGF-5, FGF-6, FGF-7, FGF-8, FGF-9, basic fibroblast growth factor, platelet-induced growth factor, transforming growth factor beta 1, acidic fibroblast growth factor, osteonectin, angiopoietin 1, angiopoietin 2, insulin-like growth factor, an antibody or antibody fragment, thymosin β-4, Nkx2.5, SV40 large T-antigen, D-Type cyclins, CDK2, dominant interfacing TSC2, p193, p53, p38, mitogen activated protein kinase (“MAPK”), cyclin A2, bck-2, GLP-1, IGF-1, chromaffin cells, collagen bioscaffold, matricelluar proteins, and tumor necrosis factor-α (“TNF-α”).

10. The biological construct of claim 9, wherein the antibody or antibody fragment has a binding affinity to one or more of the antigens selected from the group consisting of CD34 receptors, CD133 receptors, CDw90 receptors, CD 117 receptors, HLA-DR, VEGFR-1, VEGFR-2, Muc-18 (CD146), CD130, stem cell antigen (“Sca-1”), stem cell factor 1 (“SCF/c-Kit ligand”), Tie-2, and HAD-DR.

11. The biological construct of claim 2, wherein each biocompatible polymeric layer comprises a sub-layer comprising a second therapeutic agent.

12. The biological construct of claim 2, wherein
a. the biocompatible polymeric layer of the first layer comprises a first naturally-derived polymer selected from the group consisting of collagen, hyaluronan, fibrin, chitosan, and gelatin;
b. the biocompantible polymeric layer of the second layer comprises a second naturally-derived polymer selected from the group consisting of collagen, hyaluronan, fibrin, chitosan, and gelatin; and
c. the biocompatible polymeric layer of the third layer comprises a synthetic polymer selected from the group consisting of poly(l-lactic acid) (“PLLA”), poly(glycolic acid) (“PGA”), poly(lactic-co-glycolic acid) (“PLGA”), polyethylene glycol (“PEG”), polycaprolactone (“PCL”), and polyurethane (“PU”).

13. The biological construct of claim 12, wherein each layer comprises a blend of naturally-derived polymers and synthetic polymers.

14. The biological construct of claim 2, wherein each biocompatible polymeric layers comprises a polymer selected from the group consisting of poly(l-lactic acid) (“PLLA”), poly(glycolic acid) (“PGA”), poly(lactic-co-glycolic acid) (“PLGA”), polyethylene glycol (“PEG”), polycaprolactone (“PCL”), poly (N-isopropylacrilamide) (“PIPAAm”), poly(ether urethane), dacron, polytetrafluorurethane, polyurethane (“PU”), silicon, cellulose ester, collagen I, collagen III, elastin, fibronectin, fibrin, fibrinogen, laminin, hydroxyapetites, hyaluronan, glycosylaminoglycans, chitosan, or alginates.

15. The biological construct of claim 14, wherein at least one biocompatible polymeric layer comprises a blend of PLGA and PLLA in equal proportions.

16. A method of healing and reconstructing an injured tissue, comprising:
a. providing a biological construct having a polymeric biomatrix, comprising a plurality of biocompatible polymeric layers, each biocompatible polymeric layer, comprising:
i. a nanophase surface texture comprising surface crystal grain sizes of less than or equal to approximately 100 nm arranged in a pre-determined pattern designed for improved, programmable, sequential drug delivery and specific tissue remodeling that is designed to recapitulate the natural healing process; and
ii. a therapeutic agent seeded within the biocompatible polymeric layer, wherein the therapeutic agent in the biocompatible polymeric layer is selected from the group consisting of an inflammatory response agent, a proliferative agent; and a remodeling agent to correspond with a phase of wound healing and tissue remodeling; and
inserting the biological construct at a site of injury, thereby healing and reconstructing the injured tissue.

17. The method of claim 16, wherein
a. the inflammatory response agent is selected from the group consisting of a neural mitogen, a cell migration activator, a thrombin activator, a differentiation agent, a growth factor, and a trophic factor;
b. the proliferative agent is selected from the group consisting of a replication inducing agent, a stem cell mobilizing factor, an endothelial cell attractant, a second neural mitogen, a second cell migration activator, a second differentiation agent, an angiogenic agent, a second growth factor, a second trophic factor, a neuroprotective agent, a cell-cycle activator, an extracellular matrix forming agent, and a neurite outgrowth agent; and
c. the remodeling agent is selected from the group consisting of a second stem cell mobilizing factor, a second endothelial cell attractant, a second cell-cycle activator, a second neuroprotective agent, and an anti-scarring agent.

18. The method of claim 16, wherein
a. the inflammatory response agent is selected from the group consisting of epidermal growth factor (“EGF”), basic fibroblast growth factor (“bFGF”), large T-antigen, granulocyte macrophage colony-stimulating factor (“GM-CSF”), platelet derived growth factor (“PDGF”), cytokines, bone morphogenic proteins (“BMP”) inhibitors, Wnt inhibitors, and insulin-like growth factor-1 (“IGF-1”);
b. the proliferative agent is selected from the group consisting of neurogenin (“Ngn3”), glucagon like peptide (“GLP-1”), granulocyte colony-stimulating factor (“G-CSF”), colony-stimulating factor (“CSF”), CSF-1, macrophage colony-stimulating factor (“M-CSF”), c-mpl ligand, megakaryocyte growth and differentiation factor (“MGDF”), erythropoietin (“EPO”), stem cell factor (“SCF”), fms-like tyrosine kinase receptor-3 (“flt3”) ligand, vascular endothelial growth factor (“VEGF”), fibroblast growth factor (“FGF”)-3, FGF-4, FGF-5, FGF-6, FGF-7, FGF-8, FGF-9, basic fibroblast growth factor (“bFGF”), platelet-induced growth factor, transforming growth factor (“TGF”) α-1, acidic fibroblast growth factor, osteonectin, angiopoietin 1, angiopoietin 2, insulin-like growth factor, an antibody or antibody fragment, thymosin β-4, homeobox protein Nkx-2.5 (“Nkx2.5”), SV40 large T-antigen, D-type cyclins, cyclin-dependent kinase 2 (“CDK2”), dominant interfacing TSC2, p193, p53, p38, mitogen-activated protein kinase (“MAPK”), cyclin A2, bypass of kinase C protein (“bck-2”), IGF-1, EGF, large T-antigen, GM-CSF, PDGF, FGF-2, bone derived neurotrophic factor (“BDNF”), neurotrophin 3 (“NT3”), neurotrophin 4/5 (“NT4/5”), nerve growth factor (“NGF”), glial derived neurotrophic factor (“GDNF”), thyroid hormone T3, galectin, integrins, TGF-α, TGF-β, BMPs, Ang-1, and hepatic growth factor/scatter factor (“HGF/SF”), neurotrophin 4 (“NT4”), and connective tissue nutrient formula (“CTNF”); and
c. the remodeling agent is selected from the group consisting of CSF, CSF-1, G-CSF, M-CSF, c-mpl ligand, MGDF, thrombopoietin (“TPO”), EPO, SCF, flt3 ligand, VEGF, FGF-3, FGF-4, FGF-5, FGF-6, FGF-7, FGF-8, FGF-9, bFGF, platelet-induced growth factor, TGF β1, acidic fibroblast growth factor, osteonectin, angiopoietin 1, angiopoietin 2, insulin-like growth factor, an antibody or antibody fragment, thymosin β-4, Nkx2.5, SV40 large T-antigen, D-Type cyclins, CDK2, dominant interfacing TSC2, p193, p53, p38, MAPK, cyclin A2, bck-2, GLP-1, IGF-1, chromaffin cells, collagen bioscaffold, matricelluar proteins, and tumor necrosis factor-α (“TNF-α”).

19. The method of claim 16, wherein each biocompatible polymeric layer comprises a sub-layer.

20. The method of claim 19, wherein the sub-layer of the first layer comprises a cell survival agent.

21. The method of claim 16, wherein the biocompatible polymeric layer comprises a polymer selected from the group consisting of poly(l-lactic acid) (“PLLA”), poly(glycolic acid) (“PGA”), poly(lactic-co-glycolic acid) (“PLGA”), polyethylene glycol (“PEG”), polycaprolactone (“PCL”), poly (N-isopropylacrilamide) (“PIPAAm”), poly(ether urethane), dacron, polytetrafluorurethane, polyurethane (“PU”), silicon, cellulose ester, collagen I, collagen III, elastin, fibronectin, fibrin, fibrinogen, laminin, hydroxyapetites, hyaluronan, glycosylaminoglycans, chitosan, and alginates.
